(19) United States
(12) Patent Application Publication
(10) Pub. No.: US 2019/0201491 A1
(43) Pub. Date: Jul. 4, 2019

(54) BOVINE FIBROBLAST GROWTH FACTOR 21 AND KETOSIS IN DAIRY CATTLE

(71) Applicants: ELANCO US INC., Greenfield, IN (US); AMBRX, INC., La Jolla, CA (US)

(72) Inventors: Peter Connor CANNING, Indianapolis, IN (US); Shailaja SRINAGESH, San Mateo, CA (US); Anthony MANIBUSAN, San Diego, CA (US); Jason PINKSTAFF, Encinitas, CA (US); Nickolas KNUDSEN, Escondido, CA (US)

(73) Assignees: Elanco US Inc., Greenfield, IN (US); Ambrx, Inc., La Jolla, CA (US)

(21) Appl. No.: 16/325,345

(22) PCT Filed: Aug. 18, 2017
(86) PCT No.: PCT/US2017/047668

§ 371 Date: Feb. 13, 2019

Related U.S. Application Data
(60) Provisional application No. 62/377,869, filed on Aug. 22, 2016.

(51) Int. Cl. A61K 38/18; A61P 3/00; C07K 14/50; A61K 47/10

(52) U.S. Cl. CPC A61K 38/1825; A61K 47/10; C07K 14/503; A61P 3/00

(57) ABSTRACT

The present application discloses PEG modified variants of a bFGF-21 polypeptide, compositions containing a bFGF-21 polypeptide variant, and methods useful in treating and/or preventing ketosis that administer the variant or a composition containing said bFGF-21 variant.

### REFERENCE TO RELATED APPLICATIONS

This application claims the benefit of U.S. Provisional Application No. 62/377,869, filed on Aug. 22, 2016

### SEQUENCE LISTING

The instant application contains a Sequence Listing which has been submitted in ASCII format via EFS-Web and is hereby incorporated by reference in its entirety. Said ASCII copy, was created on Aug. 15, 2017 is named 204257_0027_WO_563187_SL.txt and is 15,591 bytes in size.

Disclosed are bovine fibroblast growth factor 21 (bFGF-21) variants modified with poly(ethylene glycol) (PEG), and methods of treating ketosis in dairy cattle using such molecules.

High levels of ketones, a condition called ketoacidosis, are detrimental and can be life threatening to an animal. Dairy cattle are particularly prone to ketoacidosis, also called simply ketosis. During late gestation and early lactation post-partum, a cow undergoes drastic metabolic changes and it often does not have enough available glucose to meet its energy needs, resulting in ketosis. To meet energy demands, stored fat molecules are released as non-esterified fatty acids (NEFA). Ketosis is characterized by lack of appetite, either lethargy or excitability, weight loss, decreased milk production, uncoordinated movement, immunosuppression, and neurological disorders. The cow may be at higher risk for mastitis, which can further impact milk production.

Past treatments for ketosis included an increase in the amount of glucose in the diet, administration of glucocorticoids such as dexamethasone to induce hyperglycemia, administration of insulin which can increase ketone metabolism, and stimulation of gluconeogenesis by providing propylene glycol, vitamin B12, and sources of organic phosphate. Combination therapies are also common. However, these treatments are done therapeutically to reduce ketosis symptoms after they appear. Because of detrimental effects, such treatments are not done prophylactically to prevent development of ketosis.

Fibroblast growth factor (FGF)-21 is a hormone that regulates the metabolism of glucose and lipid homeostasis. The human FGF-21 cDNA sequence was submitted to GenBank on Aug. 3, 2000 and has the Accession No. AB021975. FGF-21 functions by binding a subset of FGF receptors and the coreceptor β-Klotho. Use of human FGF-21 variants to treat obesity and diabetes in humans has been suggested, for example, in WO2013/131091, WO2013/184958, and WO2013/188182. Plasma concentrations of bFGF-21 have been found to increase at parturition and during early lactation. Schoenberg, et al., (2011) Endocrinology 152: 4652-61. However, bFGF-21 was not previously reported to affect ketosis in dairy cattle.

The wild type bFGF-21 was submitted to GenBank on Aug. 5, 2013 and has the sequence of GenBank Accession No. X1³_002695246.1. The reported sequence of the wild type bFGF-21 is:

MGWDEAKFKHLGLWVPVLAVLLLGTCRAHPIPDSSPLLQFGGQVRQRYLY
TDDAQETEAHLEIRADGTVVGAARQSPESLLELKALKPGVIQILGVKTSR
FLCQGPDGKLYGSLHFDPKACSFRELLLEDGYNVYQSETLGLPLRLPPQR
SSNRDPAPRGPARFLPLPGLPAAPPDPPGILAPEPPDVGSSDPLSMVGPS
YGRSPSYTS.

New treatments are needed for ketosis in cattle, and especially dairy cattle, particularly treatments that can be administered prophylactically or in subclinical cases of ketosis. Accordingly, as disclosed herein PEGylated bFGF-21 variants can be administered at or before parturition to reduce ketosis without detrimental effects. Modification of bFGF-21 is desired to increase its serum half-life, water solubility, bioavailability, therapeutic half-life, or circulation time, or to modulate immunogenicity, or biological activity. Such modifications can include the covalent attachment of the hydrophilic, polymer poly(ethylene glycol), abbreviated PEG. To maximize the desired properties of PEG, the total molecular weight and hydration state of the PEG polymer or polymers attached to the biologically active molecule must be sufficiently high to impart the advantageous characteristics typically associated with PEG polymer attachment, such as increased water solubility and circulating half-life, while not adversely impacting the bioactivity of the molecule to which the PEG polymer is attached.

PEG derivatives are frequently linked to biologically active molecules through reactive chemical functionalities, such as amino acid residues, the N-terminus, and/or carbohydrate moieties. WO 99/67291 discloses a process for conjugating a protein with PEG, wherein at least one amino acid residue on the protein is substituted with a synthetic amino acid and the protein is contacted with PEG under conditions sufficient to achieve conjugation to the protein.

Proteins and other molecules often have a limited number of reactive sites available for polymer attachment. The sites most suitable for modification via polymer attachment may play a significant role in receptor binding, and such sites may be necessary for retention of the biological activity of the molecule therefore making them inappropriate for polymer attachment. As a result, indiscriminate attachment of polymer chains to such reactive sites on a biologically active molecule often leads to a significant reduction or even total loss of biological activity of the polymer-modified molecule. PEG attachment can be directed to a particular position within a protein such that the PEG moiety does not interfere with the function of that protein. One method of directing PEG attachment is to introduce a synthetic amino acid into the protein sequence. The protein biosynthetic machinery of the prokaryote Escherichia coli (E. coli) can be altered in order to incorporate synthetic amino acids efficiently and with high fidelity into proteins in response to the amber codon, UAG. See, e.g., J. W. Chin et al., (2002), J. Amer. Chem. Soc. 124: 9026-9027; J. W. Chin, & P. G. Schultz, (2002), ChemBioChem 3(11): 1135-1137; J. W. Chin, et al., (2002), PNAS USA 99: 11020-11024; and, L. Wang, & P. G. Schultz, (2002), Chem. Comm., 1: 1-11. A similar method can be accomplished with the eukaryote, Saccharomyces cerevisiae (S. cerevisiae) (e.g., J. Chin et al., Science 301: 964-7 (2003)). Using this method, the synthetic amino acid para-acetylphenylalanine (pAF) can be incorporated into bFGF-21 to serve as an attachment site for PEG. See, WO 2010/011735.

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

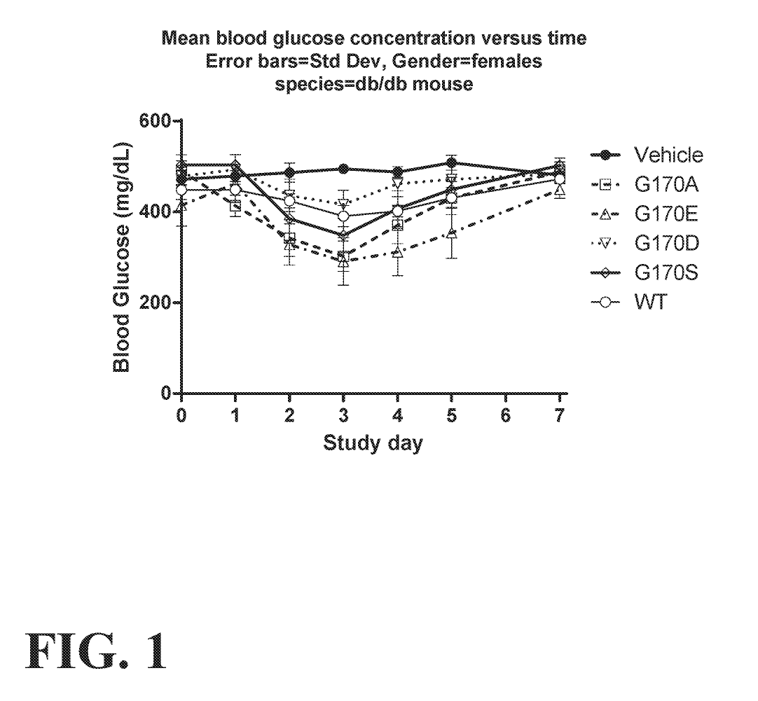

FIG. 1. The in vivo glucose-lowering activity of bFGF-21 variants having different substitutions at position G170, as demonstrated in the db/db mouse model.

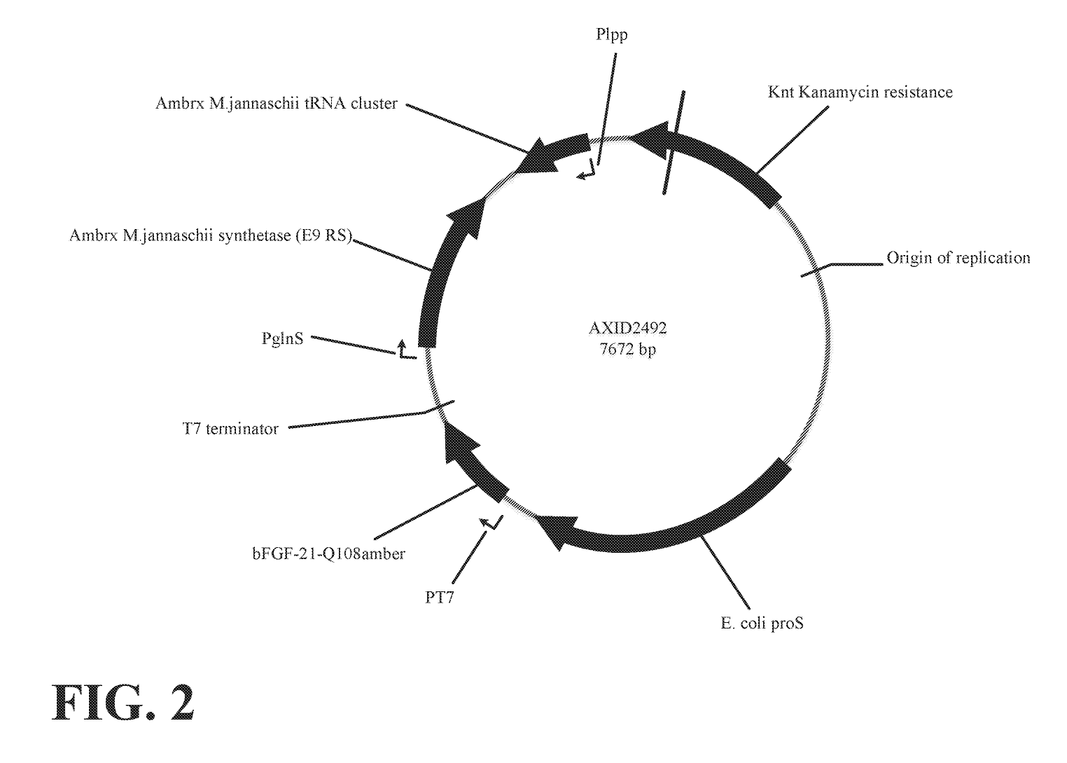

FIG. 2. Map of expression vector containing a bFGF-21 variant and the necessary genetic information to direct the biosynthetic incorporation of a synthetic amino acid at the site of an amber stop codon (UAG).

Provided here is a bFGF-21 variant having an amino acid sequence of: MHPIPDSSPLLQFGGQVRQRYLYTDDAQETEAHLEIRADGTVVGAARQSPESLLELK ALKPGVIQILGVKTSRFLCQGPDGKLYGSLHFDPKACSFRELLLEDGYNVYQSETLGL PLRLPPQRSSNRDPAPRGPARFLPLPGLPAAPPDPPGILAPEPPDVGSSDPLSMVEPSY GRSPSYTS (SEQ ID NO: 1), or

HPIPDSSPLLQFGGQVRQRYLYTDDAQETEAHLEIRADGTVVGAARQSPESLLELKAL KPGVIQILGVKTSRFLCQGPDGKLYGSLHFDPKACSFRELLLEDGYNVYQSETLGLPL RLPPQRSSNRDPAPRGPARFLPLPGLPAAPPDPPGILAPEPPDVGSSDPLSMVEPSYGR SPSYTS (SEQ ID NO: 2), in which the glycine (G) at position 170 is changed to glutamic acid (E). The G170E mutation appears in bold and underlined in SEQ ID NOs 1 and 2 above. As compared to SEQ ID NO: 1, SEQ ID NO: 2 also does not contain the methionine (M) at the start of the peptide. Reference to specific amino acids is based upon the peptide sequence without the M at the starting position, i.e. numbering begins with the amino-terminal histidine residue. A synthetic amino acid is substituted at a residue selected from G77, K91, Q108, and R131 in either of SEQ ID NOs: 1 or 2. The synthetic amino acid substituted in each of these four locations can be para-acetylphenylalanine (pAF). The bFGF-21 variants can be PEGylated at the synthetic amino acid located at one of the indicated locations (i.e., G77, K91, Q108, and R131).

Provided here is a bFGF-21 variant having an amino acid sequence of: HPIPDSSPLLQFGGQVRQRYLYTDDAQETEAHLEIRADGTVVGAARQSPESLLELKAL KPGVIQILGVKTSRFLCQGPDGKLYGSLHFDPKACSFRELLLEDGYNVY[pAF]SETLG LPLRLPPQRSSNRDPAPRGPARFLPLPGLPAAPPDPPGILAPEPPDVGSSDPLSMVEPS YGRSPSYTS (SEQ ID NO: 3), in which the glycine (G) at position 170 is changed to glutamic acid (E). The G170E mutation appears in bold and underlined in the above sequence. A synthetic amino acid, para-acetylphenylalanine (pAF) is substituted at residue Q108, The bFGF-21 variant is PEGylated at the synthetic amino acid.

The PEG moieties used can have average molecular weights between 10 kDa and 100 kDa, or between 20 kDa and 50 kDa. For example, a PEG moiety can have a molecular weight of about 20 to about 40 kDa. The PEG moiety can have a molecular weight of about 30 kDa. The PEG molecule can be a linear molecule having a molecular weight of 30 kDa. The PEG molecule can have an aminooxy group capable of reacting with an acetyl group on a synthetic amino acid. The PEG molecule can be a 30 kDa aminooxy activated linear PEG capable of forming an oxime bond with the acetyl side chain of pAF. The PEG can be for example a linear 30 kDa PEG (e.g., 30KPEG) α-methyl-ω-aminooxyethylcarbamyl, polyoxyethylene.

A pharmaceutical composition is provided that includes a PEGylated bFGF-21 variant and at least one pharmaceutically acceptable carrier, diluent, or excipient.

The PEGylated bFGF-21 variant and formulations thereof can be used in therapy. The PEGylated bFGF-21 variant can be used in the treatment of ketosis in a bovine. The PEG moieties used can have average molecular weights between 10 kDa and 100 kDa, or between 20 kDa and 50 kDa. For example, a PEG moiety can have a molecular weight of about 20 to about 40 kDa. The PEG moiety can have a molecular weight of about 30 kDa.

The PEG molecule can be a linear molecule having a molecular weight of 30 kDa. The bovine can be a dairy cow, or the bovine can be a pregnant dairy cow. The therapy can comprise administering about 20-200 μg/kg animal weight of the bFGF-21 variant to the bovine. The therapy can comprise administering about 25-100 μg/kg animal weight, or about 50 μg/kg animal weight, of the bFGF-21 variant to the bovine. The PEGylated bFGF-21 variant can be administered at least once 7 days or less prior to calving, or administered at calving. The therapy can consist of a second administration given 7 days or less after calving.

The PEGylated bFGF-21 variant can be used in the manufacture of a medicament for the treatment of ketosis in a bovine. The PEG moieties used can have average molecular weights between 10 kDa and 100 kDa, or between 20 kDa and 50 kDa. For example, a PEG moiety can have a molecular weight of about 20 to about 40 kDa. The PEG moiety can have a molecular weight of about 30 kDa. The PEG molecule can be a linear molecule having a molecular weight of 30 kDa. The bovine can be a dairy cow, or the bovine can be a pregnant dairy cow. The treatment can comprise administering about 20-200 μg/kg animal weight of the bFGF-21 variant to the bovine. The treatment can comprise administering about 25-100 μg/kg animal weight, or about 50 μg/kg animal weight, of the bFGF-21 variant to the bovine. The PEGylated bFGF-21 variant can be administered at least once 7 days or less prior to calving, or administered at calving. The treatment can consist of a second administration given 7 days or less after calving.

A method for treating ketosis in a bovine can include administering a therapeutically effective amount of the PEGylated bFGF-21 variant to the bovine in need thereof The bovine may be a dairy cow. The bovine may be a pregnant dairy cow. The therapeutically effective amount of PEGylated bFGF-21 in the method can be about 20-200 μg/kg of animal weight, about 25-100 μg/kg of animal weight, or about 50 μg/kg of animal weight. The administration of the PEGylated variant can occur at least once 7 days or less prior to calving by the pregnant cow. The administration of the PEGylated variant can occur at least twice, wherein a first administration is given at or prior to calving, and a second administration is given to the cow about 7 days or less after calving. Two administrations can be about 5 to about 28 days apart, or about 7 to 21 days apart, or about 14 days apart.

A method is provided for reducing an amount of a non-esterified fatty acid (NEFA) and/or an amount of a β-hydroxy butyric acid (BHBA) level in a bovine comprising administering the PEGylated bFGF-21 variant to the bovine in need thereof. The serum concentration of NEFA can be less than 0.6 mg/L. The serum concentration of BHBA can be less than 1.2 mg/L. The administration of the PEGylated variant can occur at least once prior to calving.

A bFGF-21 variant that encodes for the G170E substitution and an amber stop codon at position Q108 is encoded by the nucleotide sequence:

(SEQ ID NO: 4)
CATCCTATTC CTGATTCTTC TCCTCTGCTG
CAATTTGGGG GTCAGGTGCG CCAACGTTAC
CTGTACACCG ACGATGCGCA AGAAACTGAG
GCTCACCTGG AGATCCGTGC TGACGGGACT
GTCGTGGGGG CTGCCCGTCA ATCCCCAGAG
TCACTGCTGG AACTGAAAGC CCTGAAGCCT
GGGGTCATTC AGATCCTGGG CGTARAGACG
AGTCGTTTCC TGTGCCAAGG CCCTGACGGG
AAACTGTATG GCTCGCTGCA TTTTGATCCT
AAAGCTTGTA GTTTTCGCGA ACTGCTGCTG
GAAGATGGTT ACAATGTGTA TTAGAGTGAA
ACTCTGGGTC TGCCTCTGCG TCTGCCTCCT
CAACGTAGTA GCAACCGTGA CCCTGCCCCG
CGCGGTCCGG CCCGTTTTCT GCCACTGCCT
GGCCTGCCTG CTGCACCACC TGACCCACCG
GGTATTCTGG CTCCGGAACC TCCAGACGTC
GGGAGTTCAG ATCCTCTGTC GATGGTAGAA
CCGTCATACG GTCGCTCTCC TAGTTACACT
TCA.

The wild type bFGF-21 polypeptide is modified as follows. The signal sequence, which is 28 amino acids in length, is replaced with a single methionine residue and Glycine-170 is replaced with glutamic acid. The amino acid sequence of the modified polypeptide is: MHPIPDSSPLLQFGGQVRQRYLYTDDAQETEAHLEIRADGTVVGAARQSPESLLELK ALKPGVIQILGVKTSRFLCQGPDGKLYGSLHFDPKACSFRELLLEDGYNVYQSETLGL PLRLPPQRSSNRDPAPRGPARFLPLPGLPAAPPDPPGILAPEPPDVGSSDPLSMVEPSY GRSPSYTS (SEQ ID NO: 1). The bold/underlined amino acid above correspond to Q108 (numbering from the N-terminal histidine of the mature wild type form of the peptide, for example as in SEQ ID NO: 2) and a substitution of G170E respectively. Q108 may be substituted with a synthetic amino acid such as pAF. The polypeptide may be PEGylated on the pAF or other incorporated synthetic amino acid such as acetylglucosaminyl-L-serine and N-acetylglucosaminyl-L-threonine.

SEQ ID NO: 3 corresponds to bFGF-21 with the Q108pAF and G170E substitutions. HPIPDSSPLLQFGGQVRQRYLYTDDAQETEAHLEIRADGTVVGAAR QSPESLLELKAL KPGVIQILGVKTSRFLCQGPDGKLYGSLHFDPKACSFRELLLEDGYNVY[pAF]SETLG LPLRLPPQRSSNRDPAPRGPARFLPLPGLPAAPPDPPGILAPEPPDVGSSDPLSMVEPS YGRSPSYTS (SEQ ID NO: 3). The bold letters in the sequence correspond to substitutions of Q108pAF and G170E refer, respectively. The polypeptide may be PEGylated at the pAF site.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein, “about” is meant to encompass variations of ±10%, ±5%, or ±1%.

As used herein, the terms “treating,” “to treat,” or “treatment,” include inhibiting, slowing, stopping, reducing, ameliorating, or reversing the progression or severity of an existing symptom, disorder, condition, or disease. A treatment may be applied prophylactically or therapeutically.

The term “therapeutically effective amount” refers to the amount or dose of a variant as described herein, which, upon single or multiple dose administration to the subject, provides the desired treatment.

A “synthetic amino acid” refers to an amino acid that is not one of the 20 common amino acids or pyrrolysine or selenocysteine. Examples of such synthetic amino acids include, but are not limited to, para-acetylphenylalanine (pAF), acetylglucosaminyl-L-serine, and N-acetylglucosaminyl-L-threonine. For additional details on such synthetic amino acids and their incorporation and modification, see WO2010/011735 and WO2005/074650.

The bFGF-21 variants of the present invention may readily be produced in a variety of cells including mammalian cells, bacterial cells such as E. coli, Bacillus subtilis, or Pseudomonas fluorescence, and/or in fungal or yeast cells. The host cells can be cultured using techniques well known in the art. The vectors containing the polynucleotide sequences of interest (e.g., the variants of FGF-21 and expression control sequences) can be transferred into the host cell by well-known methods, which vary depending on the type of cellular host. For example, the calcium chloride transformation method is commonly utilized for prokaryotic cells, whereas calcium phosphate treatment or electroporation may be used for other eukaryotic host cells. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994).

The PEGylated bFGF-21 variants can be formulated according to known methods to prepare pharmaceutically useful compositions. A desired formulation is a stable lyophilized product that is reconstituted with an appropriate diluent or an aqueous solution of high purity with optional pharmaceutically acceptable carriers, preservatives, excipients or stabilizers [Remington, The Science and Practice of Pharmacy, 19th ed., Gennaro, ed., Mack Publishing Co., Easton, Pa. 1995].

The PEGylated bFGF-21 variant may be formulated with a pharmaceutically acceptable buffer, and the pH adjusted to provide acceptable stability, and a pH acceptable for administration. Moreover, the PEGylated bFGF-21 compositions of the present invention may be placed into a container such as a vial, a cartridge, a pen delivery device, a syringe, intravenous administration tubing or an intravenous administration bag.

The following experimental examples are illustrative of selecting non-PEGylated precursor bFGF-21 variants, generating PEGylated variants of bFGF-21, and the efficacy of a PEGylated bFGF-21 variant in regards to treating dairy cattle.

### EXAMPLE 1

The biological activity of PEGylated bFGF-21 (PEG-bFGF-21) proteins is measured with the STEADY-GLO® Elk1 luciferase reporter assay (Promega). PEG-bFGF-21 binds to beta Klotho and FGFR1c (Fibroblast Growth Factor Receptor isoform 1c) which are expressed on the cell surface of a proprietary stable cell line (HEK293), initiating a signaling cascade that results in phosphorylation of an Elk1 fusion protein. The activated (e.g., phosphorylated) Elk1 fusion protein then translocates to the cell nucleus, binds to an upstream activating sequence in a reporter cassette and drives expression of luciferase.

Luciferase is quantified by addition of substrate according to manufacturer instructions. The amount of luminescence produced is proportional to the activity of PEG-bFGF-21. The potency of the PEG-bFGF-21 variant is calculated by comparison of its half-maximal effective concentration (EC₅₀) value obtained from a 4-parameter sigmoidal fit of the dose response curve to the EC₅₀ value of a comparator protein, wild-type (WT) bFGF-21, run on the same assay plate. The maximum efficacy (Emax) of PEG-bFGF-21 is calculated by comparison of its maximum relative light units (RLU) signal to the maximum RLU signal of WT bFGF-21.

Four 30 kDa PEG (30KPEG)-bFGF-21 variants are tested for activity using the Elk1 luciferase reporter assay. Sites for the introduction of amber stop codons are selected based on an analysis of the human FGF-21 crystal structure. Selected sites are remote from the receptor binding region of FGF-21. bFGF-21 variants with a TAG codon for substituting a pAF synthetic amino acid at each selected position are generated by site-directed polymerase chain reaction (PCR) mutagenesis. Corresponding bFGF-21 pAF site variant plasmids are transformed into E. coli cells containing the expanded genetic code system components for pAF incorporation. Transformed cells are grown in media supplemented with pAF and induced to express bFGF-21 with pAF incorporated into the sites indicated. Cells are harvested and the target bFGF-21 pAF site variants are isolated and purified. An activated 30 kDa linear aminooxy-PEG is site-specifically conjugated to the incorporated pAF. PEG-bFGF-21 conjugates are purified from excess PEG and unconjugated bFGF-21 by chromatography.

Conjugation of 30 kDa PEG at the pAF substituted for Q108, G77, K91, or R131 of bFGF-21 impacted in vitro activity differentially. Table 1 summarizes the results of the PEG conjugation site experiment. Among the four tested variants, bFGF-21-Q108-30KPEG retained the most biological activity in the STEADY_GLO luciferase assay with a 4× loss in potency and the highest Emax (73%) relative to WT non-PEGylated bFGF-21. As a result, Q108 was selected as the optimal position of the four tested for pAF substitution and PEG conjugation.

TABLE 1
In Vitro Activity of PEG-bFGF-21 Site Variants

| | EC50 | Fold loss potency relative to | Emax |
|---|---|---|---|
| | (ng/mL) | WT bFGF21 | (%) |
| WT bFGF-21 | 38 | 1x | 100 |
| bFGF-21-Q108-30KPEG | 147 | 4x | 73 |
| bFGF-21-G77-30KPEG | 347 | 9x | 50 |
| bFGF-21-K91-30KPEG | 537 | 14x | 57 |
| bFGF-21-R131-30KPEG | 556 | 14x | 38 |

### EXAMPLE 2

In serum, native human FGF-21 (hFGF-21) is susceptible to proteolytic cleavage at the C-terminus, resulting in a significant loss of hFGF-21 potency. Four amino acid substitutions for the glycine at position 170 (G170) of bFGF-21 are generated to potentially prevent C-terminal clipping. The in vitro activity of the bFGF-21-Q108-30KPEG G170 variants is measured with the Elk1 luciferase reporter assay. The Elk1 luciferase assay (see Example 1) method utilizes tissue culture medium, therefore C-terminal clipping and associated activity loss of bFGF-21-Q108-30KPEG are not expected to occur in this assay. Table 2 summarizes the comparative in vitro activity of the bFGF-21-Q108-30KPEG G170 variants, generated by site-directed PCR mutagenesis and expressed in E. coli as above. The G170S and G170A variants retained the most in vitro activity, with potency losses of only lx or 2× relative to bFGF-21-Q108-30KPEG G170, respectively.

TABLE 2
In Vitro Activity of bFGF-21-Q108-30KPEG G170 Variants

| | Average Fold Loss Potency Relative to |
|---|---|
| Bovine FGF-21 Variant | bFGF-21-Q108-30KPEG |
| 30K PEG-Q108 | 1X |
| 30K PEG-Q108 G170A | 1.7X |
| 30K PEG-Q108 G170E | 3.7X |
| 30K PEG-Q108 G170D | 8.7X |
| 30K PEG-Q108 G170S | 0.8X |

### EXAMPLE 3

The in vivo activity of the four bFGF-21-Q108-30KPEG G170 variants is determined in a hyperglycemic mouse model (referred to as “db/db”) to assess the impact of each variant upon mean blood glucose levels. The bFGF-21 variants are administered at 0.75 mg/kg body weight to each of five groups of five mice per group, with a sixth group receiving vehicle only as a negative control, on day 1 of the study. Mice are 8 weeks old at the initiation of the study. Blood glucose and body weight are measured daily through day 7 of the study.

The results of this study are illustrated in FIG. 1. FIG. 1 shows that the G170A and G170E variants provided significant differences relative to negative controls. FIG. 1 plots the mean blood glucose concentration verses time (measured in study days). The error bars represent one standard deviation. As illustrated in FIG. 1, a statistically significant improvement in the glucose levels is observed in the mice administered bFGF-21-Q108-30KPEG G170 variants, as compared to the vehicle treated animals (e.g., animals treated with a buffer that has 20 mM tris, 250 mM sucrose, pH 8.5 alone).

Based upon the data above, and information regarding the efficiency of expression of each variant by the E. coli strain used for production, the variant substituting glutamic acid for glycine at position 170 was selected (SEQ ID NO: 1).

### EXAMPLE 4

A cloned cell line expressing bFGF-21Q108pAF-G170E, designated AXID2492, is isolated from a single colony isolate transformation plate that contained a modified E. coli K-12 W3110 strain containing an expression plasmid directing the expression of the engineered bFGF-21-Q108pAF-G170E. The expression plasmid contains all of the necessary genetic information to direct the biosynthetic incorporation of pAF at position 108 of the bFGF-21 amino acid sequence (FIG. 2). The form of bFGF-21-Q108pAF-G170E expressed in E. coli has an amino terminal methionine (M) which is cleaved off in the mature form of the peptide (SEQ ID NO: 3). In other organisms , for example yeast or mammalian cells, the methionine may not be present as in the amino acid sequence:

(SEQ ID NO: 3)
MHPIPDSSPLLQFGGQVRQRYLYTDDAQETEAHLEIRADGTVVGAARQSP
ESLLELKALKPGVIQILGVKTSRFLCQGPDGKLYGSLHFDPKACSFRELL
LEDGYNVY[pAF]SETLGLPLRLPPQRSSNRDPAPRGPARFLPLPGLPAA
PPDPPGILAPEPPDVGSSDPLSMVEPSYGRSPSYTS.

The bold and underlined letters in the sequence above for pAF or E refer to the site of the Q108pAF substitution or the G170E substitution, respectively.

AXID2492 includes an expression plasmid maintained in its E. coli host by growth on Kanamycin sulfate containing Luria-Bertani (LB) media (50 μg/mL kanamycin). The E. coli K-12 strain is a W3110 derivative, as described in WO2010/011735, which is genetically modified to contain a modified bFGF-21 DNA sequence with an amber stop codon (TAG) in place of the endogenous glutamine codon (CAG) at amino acid position 108. The amber stop codon is not recognized by certain bacterial strains such as Methanococcus jannaschii. A tRNA conjugated to a synthetic amino acid such as pAF can be added to the bacterial strain, thereby incorporating the synthetic amino acid into the nascent peptide being expressed in the bacteria. In addition, G170E substitution is introduced to prevent proteolytic clipping of the modified bFGF-21 protein. The nucleotide sequence of the modified bFGF-21 is:

| (SEQ ID NO: 4) | |
|---|---|
| 1 | CATCCTATTC CTGATTCTTC TCCTCTGCTG CAATTTGGGG GTCAGGTGCG CCAACGTTAC |
| 61 | CTGTACACCG ACGATGCGCA AGAAACTGAG GCTCACCTGG AGATCCGTGC TGACGGGACT |
| 121 | GTCGTGGGGG CTGCCCGTCA ATCCCCAGAG TCACTGCTGG AACTGAAAGC CCTGAAGCCT |
| 181 | GGGGTCATTC AGATCCTGGG CGTAAAGACG AGTCGTTTCC TGTGCCAAGG CCCTGACGGG |
| 241 | AAACTGTATG GCTCGCTGCA TTTTGATCCT AAAGCTTGTA GTTTTCGCGA ACTGCTGCTG |
| 301 | GAAGATGGTT ACAATGTGTA TTAGAGTGAA ACTCTGGGTC TGCCTCTGCG TCTGCCTCCT |
| 361 | CAACGTAGTA GCAACCGTGA CCCTGCCCCG CGCGGTCCGG CCCGTTTTCT GCCACTGCCT |
| 421 | GGCCTGCCTG CTGCACCACC TGACCCACCG GGTATTCTGG CTCCGGAACC TCCAGACGTC |
| 481 | GGGAGTTCAG ATCCTCTGTC GATGGTAGAA CCGTCATACG GTCGCTCTCC TAGTTACACT |
| 541 | TCA. |

The amber stop codon is underlined, and the G170E codon is double underlined.

The expression plasmid also contains genes for the tyrosyl transfer ribonucleic acid (tRNA) and tyrosine aminoacyl-tRNA synthetase (aa-RS) pair derived from Methanococcus jannaschii strain DSM 2661. This tRNA/tRNA synthetase pair is modified and genetically selected to incorporate pAF in proteins coded for by specifically engineered test genes in response to the amber stop codon. See, WO 2010/011735. High density fermentation studies are performed to confirm expression of bFGF-21-Q108pAF-G170E protein by SDS-PAGE gel analysis.

The stepwise process to produce the W3110B60 cell line and AXID2492 clone is now described. The wild-type E. coli K-12 W3110 strain is purchased from ATCC® (Catalog # 27325). To ensure proper induction of gene expression with arabinose, the cell's ability to metabolize arabinose is abolished by generalized transduction of the chromosomal copy of the araB gene with the g1-tetA gene cassette from BL21-AI strain (BL21-AI; Invitrogen, Carlsbad, Calif.) to create the W3110B2 strain. The T7 RNA polymerase gene cassette (g1-tetA) from the B2 cell line is PCR-amplified. This PCR product is integrated using homologous recombination (Kang, Y. et al., Systematic Mutagenesis of the Escherichia coli Genome, J. Bacteriology, 2004: 4921-4930) into the chromosome of the wild-type W3110 strain (ATCC® #27325). This procedure created the cell line W3110B42. Confirmation that the T7 RNA polymerase gene (g1) had been precisely integrated at araB locus is determined via PCR analysis of W3110B42 genomic DNA and sequencing of the resulting PCR product.

The fhu/tonA gene encodes a bacteriophage receptor in E. coli that allows a bacteriophage to attach to and infect E. coli. It is important to delete the fhu/tonA gene from the production host to make the host phage-resistant and thereby avoid potential contamination during the manufacturing process. From the W3110 genomic DNA, fhuA“Left” and fhuA“Right” regions are PCR-amplified. These two PCR products are digested and ligated together with the dhfr gene. The fhuA::dhfr final knockout product is PCR-amplified from the ligated product, and is integrated via homologous recombination into the chromosome of W3110B42, resulting in strain W3110B55. The presence of fhuA::dhfr in W3110B55 is sequence confirmed. The genotype of W3110B60 is F-IN(rrnD-rrnE) lambda-araB::g1-tetA fhuA::dhfr.

In a similar manner, the proS-W375R (point mutation for conversion of Tryptophan 375 to Arginine)-cat cassette is generated and incorporated into the W3110B55 chromosome through homologous recombination. This procedure created the temperature sensitive (Ts) cell line W3110B60. This point mutation in the prolyl-tRNA synthetase (proS W375R) gene confers a lethal host phenotype at temperatures >37° C. Integration of the proS-W375R-cat into the chromosome are confirmed both phenotypically (using chloramphenicol resistance) and genotypically by PCR of the W3110B60 genomic DNA and sequencing the resulting PCR product. The genotype of W3110B60 is F-IN (rrnD-rrnE) lambda-araB::g1 tetA fhuA::dhfr proSW375R-cat.

After construction of the AXID2492 clone, a single colony isolate is used to generate a Research Cell Bank (RCB). High cell density fermentation studies are performed to confirm the expression of bFGF-21-Q108pAF-G170E protein by sodium dodecyl sulfate polyacrylamide gel electrophoresis (SDS-PAGE) gel analysis. This RCB also is also characterized and confirmed using viability, DNA sequencing, phenotype analysis and phage testing experiments for identity and purity.

An amber stop codon (TAG) is inserted at the glutamine codon (CAG) corresponding to the 108 amino acid position of the mature wild type bFGF-21 protein. Glycine codon (GGT) at the 170 residue is mutated to the glutamic acid codon (GAA) to minimize proteolytic clipping of bFGF-21 protein at the C-terminus. To confirm that the cloning has proceeded as expected with no introduction of mutation(s), the entire plasmid sequence of

AXID2492 is sequenced. To prepare plasmid DNA, a 10 mL culture of LB broth containing 50 μg/mL Kanamycin sulfate is inoculated with a 10 μL stab of the glycerol stock cells and grown at 37° C., at 250 rpm overnight. The plasmid DNA sample is isolated using the QIAGEN MINIPREP Kit® according to manufacturer's instructions. The DNA sequence is confirmed after sequence analysis. The wild-type E. coli K-12 W3110 proS gene is subcloned into this vector at the BglII restriction site.

To confirm that the AXID2492 RCB is competent to produce modified bFGF-21 protein, three high cell density fermentations are performed from three separate vial thaws. The production fermenter is grown in chemically defined medium and consists of batch and fed-batch phases. The initial glycerol concentration of fermentation batch is 48 g/L. The fermentation is induced when the OD₆₀₀ reached 35 during the batch phase, and a constant feed of a solution comprised mainly of glycerol at a rate of 15 mL/L/h resumed at induction. The fermentation final wet cell densities are 172, 172, and 169 g/L for the three fermentations producing bFGF-21 Q108pAF-G170E protein. All three fermentations produced modified full length bFGF-21-Q108pAF-G170E protein upon induction. An activated 30 kDa linear aminooxy-PEG is site-specifically conjugated to the incorporated pAF at Q108. PEG-bFGF-21 conjugate is purified from excess PEG and unconjugated bFGF-21 by chromatography.

### EXAMPLE 5

The PEGylated bFGF-21 Q108-G170E variant is evaluated for its efficacy in the treatment of ketosis. Bovine (Bos taurus), non-lactating, pregnant, multiparous Holstein cows are used in the experiments below. Pre-calving, cows are fed close-up total mixed rations (TMR). The offered volume of fresh TMR feed is reduced by approximately 15 or 30% on the day of calving (Study Day 0) to induce a ketogenic state. Animals are selected for treatment approximately 7 days prior to their individual anticipated calving dates. Three experimental groups are created as illustrated in Table 3 below.

TABLE 3
Experimental Treatment Groups in Cows

| Treatment | # of Animals |
|---|---|
| Sterile Saline, 2 ml, SIDX1, SC, Day −7 SC | 14 |
| PEG bFGF-21, 50 μg/kg, SIDx1, Day −7, SC | 14 |
| PEG bFGF-21, 25 μg/kg, SIDx1, Day 0, SC | 14 |

Cows in treatment groups 1 and 2 receive their individual treatments on Day −7. Cows in treatment group 3 receive their individual treatments on Day 0. The treatment with the bFGF-21 PEGylated variant having the pAF substitution at Q108 and the stabilizing G170E mutation is administered by a subcutaneous injection in the pre-scapular region of the neck according to the amount listed in Table 3 based on the treatment group to which the cow is assigned. Physical exams conducted by a veterinarian including a review of all major body systems, rectal temperatures, heart and respiration rates, and bodyweights are performed on all animals on Days −7, 0, 7, 14, and 21.

Blood samples (approximately 5 mL) are obtained from the tail vein after removing fecal material using a syringe with a 20 gauge needle on study days −7, 0 (day of calving), 3, 7, 10, 14, 21. Blood samples are collected prior to feeding each day in a manner, which minimizes animal stress. Blood samples are allowed to clot. Serum is harvested by centrifugation. Serum samples are utilized for the detection of non-esterified fatty acid (NEFA) and β-hydroxy butyric acid (BHBA) levels. A cow is identified as clinically ketotic if:
(1) the serum BHBA levels are ≥12 mg/dL and NEFA levels≥0.6 mEq/L at any time point after calving;
(2) the serum BHBA levels≥12 mg/dL at any time point after calving; and/or
(3) the serum NEFA levels≥0.6 mEq/L at any time point after calving is considered ketotic.
   The ketotic state is also determined at each time point of days 3, 7, and 10. A dose level of PEG bFGF-21 is considered efficacious if a statistically significant (p<0.1) reduction in the incidence relative to the control group.

The statistical analysis is performed by using SAS® software (Version 9.2 or newer, SAS® Institute Inc., Cary, N.C., USA). The level of significance is set to α=0.10 for the statistical analysis of the key efficacy endpoints. One-sided tests are used for comparing the bFGF-21 variant's group means to the control mean within an analysis. The ketosis incidence rate date is analysed using Fisher's Exact test, an option of Proc Freq of the SAS® system.

The NEFA and BHBA levels are analyzed using a repeated measures analysis of variance or covariance with the mean of the pretreatment observations (BASELINE) being considered as a possible covariate for each variable.

Comparisons of treatments within each day is made at α=0.10 if there is a significant treatment by day interaction (α=0.10).

Table 4 show the impact of the PEG bFGF-21-Q108pAF-G170E variant upon daily mean serum non-esterified fatty acid (NEFA) levels.

TABLE 4
Statistical analysis of the impact of PEG bFGF-21 upon mean
daily NEFA levels
Analysis of NEFA Levels

| | NEFA (Probt¹) | | | | |
|---|---|---|---|---|---|
| Pairwise Comparisons | Day 3 | Day 7 | Day 10 | Day 14 | Day 21 |
| Saline vs. bFGF D-7 | 0.0440* | 0.0278* | 0.6337 | 0.4472 | 0.3737 |
| Saline vs. bFGF D0 | 0.0008* | 0.2834 | 0.6146 | 0.5314 | 0.7474 |
| bFGF D-7 vs. bFGF D0 | 0.1303 | 0.2668 | 0.9689 | 0.9178 | 0.5915 |

¹Probability value for the Student's t-test.

Serum NEFA levels peaked between 3 and 7 days after calving for animals in all three treatment groups. Administration of PEG bFGF-21 7 days prior to calving or on the day of calving significantly reduced NEFA serum levels on Day 3 relative to the saline control. The Day 7 post-calving value is also significantly reduced relative to the saline control. The responses of the animals are not significantly different for the two different PEG bFGF-21 variant dosing regimens.

Results of the β-hydroxy butyric acid (BHBA) assays indicated that all animals in all three treatment groups exhibit serum BHBA below 12 mg/dL prior to calving and on the day of calving. Peak serum BHBA levels are observed on Day 7 for the saline controls. Animals that are treated with PEG bFGF-21 on the day of calving exhibited a trend towards reduced serum BHBA levels relative to the saline controls, but these differences are not statistically significant. Animals that are treated with the PEG bFGF-21 variant on Day -7 exhibit similar serum levels of BHBA on Days 0-10 post-calving relative to the saline controls and a statistically non-significant trend towards somewhat higher levels on Days 14 and 21.

Approximately 85% of the cows treated with saline had serum NEFA levels≥0.6 mEq/L on the day of calving; this percentage increased to 100% by Day 3 post-calving. Approximately 65% of the cows treated with the PEG bFGF-21 variant on Day -7 had serum NEFA levels≥0.6 mEq/L on the day of calving compared to the cows treated on Day 0 which exhibited a similar percentage to the saline controls. On Day 14, the percentage of treated cows with serum NEFA values≥0.6 mEq/L is significantly (p=0.0302) higher than the saline control animals. There are no significant differences between the PEG bFGF-21 variant treatment groups.

None of the animals in the study exhibited elevated serum BHBA levels prior to calving or on the day of calving. Eighty-six percent of the animals that are treated with saline had serum BHBA levels≥12 mg/dL on Days 3 and 7 post-calving, and then BHBA levels gradually declined during the remainder of the study. Animals treated with PEGylated bFGF-21 on seven days prior to calving exhibited serum BHBA levels that are not significantly different from the saline controls at all sampling time points. In contrast, cows treated with PEGylated bFGF-21 on the day of calving exhibited decreased serum BHBA levels relative to the saline controls at all sampling time points. The difference in serum BHBA levels between the saline controls and animals treated with PEG bFGF-21 on Day 0 is statistically significant (p=0.0472). The rate that BHBA levels declined in the animals treated with the PEG bFGF-21 variant on Day 0 also appears to be somewhat faster during the first 10 days post-calving relative to the saline controls.

The incidence of abnormal daily health observations in each treatment group is monitored. The abnormal health observations are observed across treatments and are typical of those normally observed in transition dairy cows and occurred at a similar frequency to what is normally observed in a commercial dairy.

Animals in all treatment groups have a typical increase in milk production over the course of the study. There are no statistically significant differences in milk production between treatment groups (p=0.3579).

The results of this study suggest that a single administration of the PEG bFGF-21 variant either 7 days prior to calving or the day of calving has a small impact upon serum NEFA levels relative to the saline controls. The percentage of cows with serum NEFA levels at or above the threshold that is typically used to define ketosis (NEFA>0.6 mEq/L) is not significantly different between all three treatment groups. It is possible the combination of using over-conditioned cows and restricting their access to feed during the early stage of lactation overwhelms the ability of the cows to respond to the protein in time to influence the NEFA levels.

The results demonstrate that the serum BHBA levels do not increase until 3 days after calving. Animals treated with the PEG bFGF-21 Q108-G170E variant on the day of calving exhibited a trend towards a smaller increase in serum BHBA levels than the saline controls and this trend is evident throughout the duration of the study. Similarly, the percentage of cows treated with the PEGylated bFGF-21 variant on the day of calving with elevated serum BHBA levels tends to be lower than the saline controls during the first two weeks after calving. In contrast, administration of the PEG bFGF-21 variant 7 days prior to calving did not alter serum BHBA levels or the percentage of ketotic animals relative to the saline controls.

Administration of the PEGylated bFGF-21 variant had no significant impact upon milk production. In this study, administration of PEGylated bFGF-21 likely limits the cow's ability to compensate for their negative energy balance by metabolizing fat reserves in the liver. In addition, as feed is restricted during the first 3 weeks of lactation in this study, the cows could not increase their food consumption. Therefore, the lack of increased milk production may be an artifact of the experimental conditions. However, it can be concluded that administration of the PEGylated bFGF-21 variant has no negative impact upon milk production.

The set of 42 cows selected for the experiments above (42 Holstein females, ranging in weight each from 629-905 kg on Day −7) are representative of the target dairy cow population. Based on the Day −7 or Day −1 body weights (depending on treatment group) and the outcome of the above experiments, the actual dose of the bFGF-21-Q108pAF-30KPEG-G170E administered once subcutaneously is about 50 μg/kg weight of animal. There are no abnormal clinical observations observed related to the treatment with the bFGF-21-Q108pAF-30KPEG-G170E.

SEQUENCE LISTING

SEQ ID NO: 1 bFGF-21 w/single M as single sequence and G170E mutation
(no internal stop codon)
MHPIPDSSPLLQFGGQVRQRYLYTDDAQETEAHLEIRADGTVVGAARQSPESLLELKALKPGVIQILG
VKTSRFLCQGPDGKLYGSLHFDPKAGSFRELLLEDGYNVYQSETLGLPLRLPPQRSSNRDPAPRGPAR
FLPLPGLPAAPPDPPGILAPEPPDVGSSDPLSMVEPSYGRSPSYTS
SEQ ID NO: 2 SEQ ID NO: 1 without M
HPIPDSSPLLQFGGQVRQRYLYTDDAQETEAHLEIRADGTVVGAARQSPESLLELKALKPGVIQILGVKTSRFLC
QGPDGKLYGSLHFDPKACSFRELLLEDGYNVYQSETLGLPLRLPPQRSSNRDPAPRGPARFLPLPGLPAAPPDPP
GILAPEPPDVGSSDPLSMVEPSYGRSPSYTS
SEQ ID NO: 3 Stop at Q108, G170E, no M
HPIPDSSPLLQFGGQVRQRYLYTDDAQETEAHLEIRADGTVVGAARQSPESLLELKALKPGVIQILGVKTSRFLC
QGPDGKLYGSLHFDPKACSFRELLLEDGYNVYpAFSETLGLPLRLPPQRSSNRDPAPRGPARFLPLPGLPAAPPD
PPGILAPEPPDVGSSDPLSMVEPSYGRSPSYTS
SEQ ID NO: 4 nucleotide sequence for peptide of SEQ ID NO: 3
CATCCTATTC CTGATTCTTC TCCTCTGCTG CAATTTGGGG GTCAGGTGCG CCAACGTTAC
CTGTACACCG ACGATGCGCA AGAAACTGAG GCTCACCTGG AGATCCGTGC TGACGGGACT
GTCGTGGGGG CTGCCCGTCA ATCCCCAGAG TCACTGCTGG AACTGAAAGC CCTGAAGCCT
GGGGTCATTC AGATCCTGGG CGTAAAGACG AGTCGTTTCC TGTGCCAAGG CCCTGACGGG
AAACTGTATG GCTCGCTGCA TTTTGATCCT AAAGCTTGTA GTTTTCGCGA ACTGCTGCTG
GAAGATGGTT ACAATGTGTA TTAGAGTGAA ACTCTGGGTC TGCCTCTGCG TCTGCCTCCT
CAACGTAGTA GCAACCGTGA CCCTGCCCCG CGCGGTCCGG CCCGTTTTCT GCCACTGCCT
GGCCTGCCTG CTGCACCACC TGACCCACCG GGTATTCTGG CTCCGGAACC TCCAGACGTC
GGGAGTTCAG ATCCTCTGTC GATGGTAGAA CCGTCATACG GTCGCTCTCC TAGTTACACT
TCA
SEQ ID NO: 5 nucleotide sequence encoding SEQ ID NO: 1
ATGCATCCTATTCCTGATTCTTCTCCTCTGCTGCAATTTGGGGGTCAGGTGCGCCAACGT
TACCTGTACACCGACGATGCGCAAGAAACTGAGGCTCACCTGGAGATCCGTGCTGACGGG
ACTGTCGTGGGGGCTGCCCGTCAATCCCCAGAGTCACTGCTGGAACTGAAAGCCCTGAAG
CCTGGGGTCATTCAGATCCTGGGCGTAAAGACGAGTCGTTTCCTGTGCCAAGGCCCTGAC
GGGAAACTGTATGGCTCGCTGCATTTTGATCCTAAAGCTTGTAGTTTTCGCGAACTGCTG
CTGGAAGATGGTTACAATGTGTATCAGAGTGAAACTCTGGGTCTGCCTCTGCGTCTGCCT
CCTCAACGTAGTAGCAACCGTGACCCTGCCCCGCGCGGTCCGGCCCGTTTTCTGCCACTG
CCTGGCCTGCCTGCTGCACCACCTGACCCACCGGGTATTCTGGCTCCGGAACCTCCAGAC
GTCGGGAGTTCAGATCCTCTGTCGATGGTAGAACCGTCATACGGTCGCTCTCCTAGTTAC
ACTTCATAA
SEQ ID NO: 6 amber stop at G77
HPIPDSSPLLQFGGQVRQRYLYTDDAQETEAHLEIRADGTVVGAARQSPESLLELKALK
PGVIQILGVKTSRFLCQ*PDGKLYGSLHFDPKACSFRELLLEDGYNVYQSETLGLPLRLP
PQRSSNRDPAPRGPARFLPLPGLPAAPPDPPGILAPEPPDVGSSDPLSMVEPSYGRSPSYTS
* = pAF
SEQ ID NO: 7 nucleotide sequence encoding SEQ ID NO: 6
ATGCATCCTATTCCTGATTCTTCTCCTCTGCTGCAATTTGGGGGTCAGGTGCGCCAACGT
TACCTGTACACCGACGATGCGCAAGAAACTGAGGCTCACCTGGAGATCCGTGCTGACGGG
ACTGTCGTGGGGGCTGCCCGTCAATCCCCAGAGTCACTGCTGGAACTGAAAGCCCTGAAG
CCTGGGGTCATTCAGATCCTGGGCGTAAAGACGAGTCGTTTCCTGTGCCAATAGCCTGAC
GGGAAACTGTATGGCTCGCTGCATTTTGATCCTAAAGCTTGTAGTTTTCGCGAACTGCTG
CTGGAAGATGGTTACAATGTGTATCAGAGTGAAACTCTGGGTCTGCCTCTGCGTCTGCCT
CCTCAACGTAGTAGCAACCGTGACCCTGCCCCGCGCGGTCCGGCCCGTTTTCTGCCACTG
CCTGGCCTGCCTGCTGCACCACCTGACCCACCGGGTATTCTGGCTCCGGAACCTCCAGAC
GTCGGGAGTTCAGATCCTCTGTCGATGGTAGAACCGTCATACGGTCGCTCTCCTAGTTAC
ACTTCATAA
SEQ ID NO: 8 amber stop at K91
HPIPDSSPLLQFGGQVRQRYLYTDDAQETEAHLEIRADGTVVGAARQSPESLLELKALKPGVIQILGVKTSRFLC
QGPDGKLYGSLHFDP*ACSFRELLLEDGYNVYQSETLGLPLRLPPQRSSNRDPAPRGPARFLPLPGLPAAPPDPP
GILAPEPPDVGSSDPLSMVEPSYGRSPSYTS
* = pAF
SEQ ID NO: 9 nucleotide sequence encoding SEQ ID NO: 8
ATGCATCCTATTCCTGATTCTTCTCCTCTGCTGCAATTTGGGGGTCAGGTGCGCCAACGT
TACCTGTACACCGACGATGCGCAAGAAACTGAGGCTCACCTGGAGATCCGTGCTGACGGG
ACTGTCGTGGGGGCTGCCCGTCAATCCCCAGAGTCACTGCTGGAACTGAAAGCCCTGAAG
CCTGGGGTCATTCAGATCCTGGGCGTAAAGACGAGTCGTTTCCTGTGCCAAGGCCCTGAC
GGGAAACTGTATGGCTCGCTGCATTTTGATCCTTAGGCTTGTAGTTTTCGCGAACTGCTG
CTGGAAGATGGTTACAATGTGTATCAGAGTGAAACTCTGGGTCTGCCTCTGCGTCTGCCT
CCTCAACGTAGTAGCAACCGTGACCCTGCCCCGCGCGGTCCGGCCCGTTTTCTGCCACTG
CCTGGCCTGCCTGCTGCACCACCTGACCCACCGGGTATTCTGGCTCCGGAACCTCCAGAC
GTCGGGAGTTCAGATCCTCTGTCGATGGTAGAACCGTCATACGGTCGCTCTCCTAGTTAC
ACTTCATAA
SEQ ID NO: 10 amber stop at R131
HPIPDSSPLLQFGGQVRQRYLYTDDAQETEAHLEIRADGTVVGAARQSPESLLELKALKPGVIQILGVKTSRFLC
QGPDGKLYGSLHFDPKACSFRELLLEDGYNVYQSETLGLPLRLPPQRSSNRDPAP*GPARFLPLPGLPAAPPDPP
GILAPEPPDVGSSDPLSMVEPSYGRSPSYTS
* = pAF
SEQ ID NO: 11 nucleotide sequence encoding SEQ ID NO: 10
ATGCATCCTATTCCTGATTCTTCTCCTCTGCTGCAATTTGGGGGTCAGGTGCGCCAACGT
TACCTGTACACCGACGATGCGCAAGAAACTGAGGCTCACCTGGAGATCCGTGCTGACGGG
ACTGTCGTGGGGGCTGCCCGTCAATCCCCAGAGTCACTGCTGGAACTGAAAGCCCTGAAG
CCTGGGGTCATTCAGATCCTGGGCGTAAAGACGAGTCGTTTCCTGTGCCAAGGCCCTGAC
GGGAAACTGTATGGCTCGCTGCATTTTGATCCTAAAGCTTGTAGTTTTCGCGAACTGCTG
CTGGAAGATGGTTACAATGTGTATCAGAGTGAAACTCTGGGTCTGCCTCTGCGTCTGCCT
CCTCAACGTAGTAGCAACCGTGACCCTGCCCCGTAGGGTCCGGCCCGTTTTCTGCCACTG
CCTGGCCTGCCTGCTGCACCACCTGACCCACCGGGTATTCTGGCTCCGGAACCTCCAGAC
GTCGGGAGTTCAGATCCTCTGTCGATGGTAGAACCGTCATACGGTCGCTCTCCTAGTTAC
ACTTCATAA

1. A bovine fibroblast growth factor 21 (bFGF-21) variant consisting of a sequence of:
(SEQ ID NO: 3)
HPIPDSSPLLQFGGQVRQRYLYTDDAQETEAHLEIRADGTVVGAARQSPE
SLLELKALKPGVIQILGVKTSRFLCQGPDGKLYGSLHFDPKACSFRELLL
EDGYNVY[pAF]SETLGLPLRLPPQRSSNRDPAPRGPARFLPLPGLPAAP
PDPPGILAPEPPDVGSSDPLSMVEPSYGRSPSYTS,
and
wherein a para-acetylphenylalanine (pAF) synthetic amino acid present at position 108 is covalently attached to a poly(ethylene glycol) (PEG).

2. The bFGF-21 variant of claim 1, wherein the PEG has a molecular weight of about 10 kDa to about 100 kDa.

3. The bFGF-21 variant of claim 1, wherein the PEG has a molecular weight of about 20 kDa to about 50 kDa.

4. The bFGF-21 variant of claim 1, wherein the PEG has a molecular weight of about 30 kDa.

5. The bFGF-21 variant of claim 1, wherein the PEG is linear.

6. A bovine fibroblast growth factor 21 (bFGF-21) variant consisting of a sequence of:
(SEQ ID NO: 3)
HPIPDSSPLLQFGGQVRQRYLYTDDAQETEAHLEIRADGTVVGAARQSPE
SLLELKALKPGVIQILGVKTSRFLCQGPDGKLYGSLHFDPKACSFRELLL
EDGYNVY[pAF]SETLGLPLRLPPQRSSNRDPAPRGPARFLPLPGLPAAP
PDPPGILAPEPPDVGSSDPLSMVEPSYGRSPSYTS,
and
wherein a pAF synthetic amino acid present at position 108 is covalently attached to a 30 kDa linear PEG.

7. A pharmaceutical composition comprising the bFGF-21 variant of claim 1 6, and at least one pharmaceutically acceptable carrier, diluent, or excipient.

8. (canceled)

9. A method of treating a symptom, disorder, condition, or disease in a bovine comprising administering to the bovine a bFGF-21 variant of claim 1.

10. A method of treating ketosis in a bovine comprising administering to the bovine a bFGF-21 variant of claim 1.

11. A method for treating ketosis in a bovine comprising administering a therapeutically effective amount of the composition of claim 7 to the bovine in need thereof.

12. The method of claim 11, wherein the bovine is a dairy cow.

13. The method of claim 12, wherein the dairy cow is a pregnant dairy cow.

14. The method of claim 11, wherein the therapeutically effective amount of bFGF-21 is about 20-200 μg/kg animal weight.

15. The method of claim 11, wherein the therapeutically effective amount of bFGF-21 is about 25-100 μg/kg animal weight.

16. The method of claim 11, wherein the therapeutically effective amount of bFGF-21 is about 50 μg/kg animal weight.

17. The method of claim 11, wherein the administering occurs at least once 7 days or less prior to calving.

18. The method of claim 11, wherein the administering occurs at calving.

19. The method of claim 17, further comprising a second administration 7 days or less after calving.

20. A method for reducing a serum concentration of non-esterified fatty acid (NEFA) and/or β-hydroxy butyric acid (BHBA) levels in a bovine comprising administering the bFGF-21 variant of claim 1 to the bovine in need thereof.

21. A nucleic acid encoding a bovine fibroblast growth factor 21 (bFGF-21) variant, wherein the nucleic acid comprises a nucleotide sequence of:
(SEQ ID NO: 4)
CATCCTATTC CTGATTCTTC TCCTCTGCTG
CAATTTGGGG GTCAGGTGCG CCAACGTTAC
CTGTACACCG ACGATGCGCA AGAAACTGAG
GCTCACCTGG AGATCCGTGC TGACGGGACT
GTCGTGGGGG CTGCCCGTCA ATCCCCAGAG
TCACTGCTGG AACTGAAAGC CCTGAAGCCT
GGGGTCATTC AGATCCTGGG CGTAAAGACG
AGTCGTTTCC TGTGCCAAGG CCCTGACGGG
AAACTGTATG GCTCGCTGCA TTTTGATCCT
AAAGCTTGTA GTTTTCGCGA ACTGCTGCTG
GAAGATGGTT ACAATGTGTA TTAGAGTGAA
ACTCTGGGTC TGCCTCTGCG TCTGCCTCCT
CAACGTAGTA GCAACCGTGA CCCTGCCCCG
CGCGGTCCGG CCCGTTTTCT GCCACTGCCT
GGCCTGCCTG CTGCACCACC TGACCCACCG
GGTATTCTGG CTCCGGAACC TCCAGACGTC
GGGAGTTCAG ATCCTCTGTC GATGGTAGAA
CCGTCATACG GTCGCTCTCC TAGTTACACT
TCA.

22. The method according to claim 10, wherein the bovine is a dairy cow.

23. The method according to claim 22, wherein the dairy cow is a pregnant dairy cow.

24. The method according to claim 10, wherein the bFGF-21 variant is to be administered at a dose of 20-200 μg/kg animal weight.

25. The method according to claim 24, wherein the bFGF-21 variant is to be administered at a dose of 25-100 μg/kg animal weight.

26. The method according to claim 25, wherein the bFGF-21 variant is to be administered at a dose of about 50 μg/kg animal weight.

27. The method according to claim 10, wherein the bFGF-21 variant is to be administered at least once 7 days or less prior to calving.

28. The method according to claim 27, wherein the bFGF-21 variant is administered at calving.

29. The method according to claim 27, wherein the bFGF-21 variant is to be administered a second time 7 days or less after calving.


SEQUENCE LISTING (11 sequences)

SEQ ID NO: 1 - protein, 182 aa, Artificial Sequence
  Synthetic polypeptide - bFGF-21 with       amino-terminal Met and G170E mutation wherein numbering begins       with the first amino-terminal histidine residue
MHPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYQSETLGLPLRLPPQRSSNRDPAPRGPARFLPLPGLPAAPPDPPGILAPEPPDVGSSDPLSMVEPSYGRSPSYTS

SEQ ID NO: 2 - protein, 181 aa, Artificial Sequence
  Synthetic polypeptide - bFGF-21 with G170E       mutation
HPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYQSETLGLPLRLPPQRSSNRDPAPRGPARFLPLPGLPAAPPDPPGILAPEPPDVGSSDPLSMVEPSYGRSPSYTS

SEQ ID NO: 3 - protein, 181 aa, Artificial Sequence
  Synthetic polypeptide - bFGF-21 with Q108pAF       and G170E
  MISC_FEATURE (108)..(108): Xaa is para-acetylphenylalanine (pAF)
HPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYXSETLGLPLRLPPQRSSNRDPAPRGPARFLPLPGLPAAPPDPPGILAPEPPDVGSSDPLSMVEPSYGRSPSYTS

SEQ ID NO: 4 - DNA, 543 bp, Artificial Sequence
  Synthetic polynucleotide - encodes SEQ ID NO 3
catcctattcctgattcttctcctctgctgcaatttgggggtcaggtgcgccaacgttacctgtacaccgacgatgcgcaagaaactgaggctcacctggagatccgtgctgacgggactgtcgtgggggctgcccgtcaatccccagagtcactgctggaactgaaagccctgaagcctggggtcattcagatcctgggcgtaaagacgagtcgtttcctgtgccaaggccctgacgggaaactgtatggctcgctgcattttgatcctaaagcttgtagttttcgcgaactgctgctggaagatggttacaatgtgtattagagtgaaactctgggtctgcctctgcgtctgcctcctcaacgtagtagcaaccgtgaccctgccccgcgcggtccggcccgttttctgccactgcctggcctgcctgctgcaccacctgacccaccgggtattctggctccggaacctccagacgtcgggagttcagatcctctgtcgatggtagaaccgtcatacggtcgctctcctagttacacttca

SEQ ID NO: 5 - DNA, 549 bp, Artificial Sequence
  Synthetic polynucleotide - encodes SEQ ID NO 1
atgcatcctattcctgattcttctcctctgctgcaatttgggggtcaggtgcgccaacgttacctgtacaccgacgatgcgcaagaaactgaggctcacctggagatccgtgctgacgggactgtcgtgggggctgcccgtcaatccccagagtcactgctggaactgaaagccctgaagcctggggtcattcagatcctgggcgtaaagacgagtcgtttcctgtgccaaggccctgacgggaaactgtatggctcgctgcattttgatcctaaagcttgtagttttcgcgaactgctgctggaagatggttacaatgtgtatcagagtgaaactctgggtctgcctctgcgtctgcctcctcaacgtagtagcaaccgtgaccctgccccgcgcggtccggcccgttttctgccactgcctggcctgcctgctgcaccacctgacccaccgggtattctggctccggaacctccagacgtcgggagttcagatcctctgtcgatggtagaaccgtcatacggtcgctctcctagttacacttcataa

SEQ ID NO: 6 - protein, 181 aa, Artificial Sequence
  Synthetic polypeptide - bFGF-21 with G77pAF and      G170E mutation
  MISC_FEATURE (77)..(77): Xaa is para-acetylphenylalanine (pAF)
HPIPDSSPLLQFGGQVRQRYLYTDDAQETEAHLEIRADGTVVGAARQSPESLLELKALKPGVIQILGVKTSRFLCQXPDGKLYGSLHFDPKACSFRELLLEDGYNVYQSETLGLPLRLPPQRSSNRDPAPRGPARFLPLPGLPAAPPDPPGILAPEPPDVGSSDPLSMVEPSYGRSPSYTS

SEQ ID NO: 7 - DNA, 549 bp, Artificial Sequence
  Synthetic polynucleotide - encodes SEQ ID NO 6
atgcatcctattcctgattcttctcctctgctgcaatttgggggtcaggtgcgccaacgttacctgtacaccgacgatgcgcaagaaactgaggctcacctggagatccgtgctgacgggactgtcgtgggggctgcccgtcaatccccagagtcactgctggaactgaaagccctgaagcctggggtcattcagatcctgggcgtaaagacgagtcgtttcctgtgccaatagcctgacgggaaactgtatggctcgctgcattttgatcctaaagcttgtagttttcgcgaactgctgctggaagatggttacaatgtgtatcagagtgaaactctgggtctgcctctgcgtctgcctcctcaacgtagtagcaaccgtgaccctgccccgcgcggtccggcccgttttctgccactgcctggcctgcctgctgcaccacctgacccaccgggtattctggctccggaacctccagacgtcgggagttcagatcctctgtcgatggtagaaccgtcatacggtcgctctcctagttacacttcataa

SEQ ID NO: 8 - protein, 181 aa, Artificial Sequence
  Synthetic polypeptide - bFGF-21 with K91pAF and      G170E mutation
  MISC_FEATURE (91)..(91): Xaa is para-acetylphenylalanine (pAF)
HPIPDSSPLLQFGGQVRQRYLYTDDAQETEAHLEIRADGTVVGAARQSPESLLELKALKPGVIQILGVKTSRFLCQGPDGKLYGSLHFDPXACSFRELLLEDGYNVYQSETLGLPLRLPPQRSSNRDPAPRGPARFLPLPGLPAAPPDPPGILAPEPPDVGSSDPLSMVEPSYGRSPSYTS

SEQ ID NO: 9 - DNA, 549 bp, Artificial Sequence
  Synthetic polynucleotide - encodes SEQ ID NO 8
atgcatcctattcctgattcttctcctctgctgcaatttgggggtcaggtgcgccaacgttacctgtacaccgacgatgcgcaagaaactgaggctcacctggagatccgtgctgacgggactgtcgtgggggctgcccgtcaatccccagagtcactgctggaactgaaagccctgaagcctggggtcattcagatcctgggcgtaaagacgagtcgtttcctgtgccaaggccctgacgggaaactgtatggctcgctgcattttgatccttaggcttgtagttttcgcgaactgctgctggaagatggttacaatgtgtatcagagtgaaactctgggtctgcctctgcgtctgcctcctcaacgtagtagcaaccgtgaccctgccccgcgcggtccggcccgttttctgccactgcctggcctgcctgctgcaccacctgacccaccgggtattctggctccggaacctccagacgtcgggagttcagatcctctgtcgatggtagaaccgtcatacggtcgctctcctagttacacttcataa

SEQ ID NO: 10 - protein, 181 aa, Artificial Sequence
  Synthetic polypeptide - bFGF-21 with R131pAF       and G170E mutation
  MISC_FEATURE (131)..(131): Xaa is para-acetylphenylalanine (pAF)
HPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYQSETLGLPLRLPPQRSSNRDPAPXGPARFLPLPGLPAAPPDPPGILAPEPPDVGSSDPLSMVEPSYGRSPSYTS

SEQ ID NO: 11 - DNA, 549 bp, Artificial Sequence
  Synthetic polynucleotide - encodes       SEQ ID NO 10
atgcatcctattcctgattcttctcctctgctgcaatttgggggtcaggtgcgccaacgttacctgtacaccgacgatgcgcaagaaactgaggctcacctggagatccgtgctgacgggactgtcgtgggggctgcccgtcaatccccagagtcactgctggaactgaaagccctgaagcctggggtcattcagatcctgggcgtaaagacgagtcgtttcctgtgccaaggccctgacgggaaactgtatggctcgctgcattttgatcctaaagcttgtagttttcgcgaactgctgctggaagatggttacaatgtgtatcagagtgaaactctgggtctgcctctgcgtctgcctcctcaacgtagtagcaaccgtgaccctgccccgtagggtccggcccgttttctgccactgcctggcctgcctgctgcaccacctgacccaccgggtattctggctccggaacctccagacgtcgggagttcagatcctctgtcgatggtagaaccgtcatacggtcgctctcctagttacacttcataa